(19) United States
(12) Patent Application Publication
(10) Pub. No.: US 2007/0184094 A1
(43) Pub. Date: Aug. 9, 2007

(54) Method for producing a metallic temporary tattoo

(76) Inventor: Thomas D. Williams, Waxhaw, NC (US)

Correspondence Address: AKERMAN SENTERFITT, P.O. BOX 3188, WEST PALM BEACH, FL, 33402-3188, US

(21) Appl. No.: 11/349,849

(22) Filed: Feb. 8, 2006

(51) Int. Cl. A61K 9/70; A61K 8/19

(52) U.S. Cl. 424/443; 424/63

(57) ABSTRACT

A method of creating a temporary tattoo is described. The method includes creating a template for the temporary tattoo, which may be formed of vinyl. The template is applied to the skin of a person. Skin safe adhesive is applied to the skin within the template, and metal leaf is applied to the adhesive layer on the skin within the template. The template is removed to reveal a metal leaf tattoo on the skin. The metal leaf may be platinum, gold, silver, copper, aluminum or colored metal leaf.

### CROSS-REFERENCE TO RELATED APPLICATIONS

Not applicable.

### FIELD OF THE INVENTION

This invention is directed generally to methods for producing temporary tattoos, and specifically to methods for producing metallic leaf tattoos.

### BACKGROUND

The formation of temporary tattoos to decorate skin can be carried out using a number of different methods. For example, henna dye may be applied to the skin such that it is absorbed into the skin. Henna will produce a temporary tattoo that can remain on the skin for several weeks, but it is difficult and time-consuming to apply. Alternatively, the skin may be painted with makeup or body paint, but such paint typically lasts several hours at most, and is likely to become smudged or otherwise rubbed off quickly. Stickers may also be applied to the skin for a temporary decoration, but stickers may only stay in place for several hours.

Stencils may be used for applying decorations to the skin. U.S. Pat. No. 5,836,998 to Mueller et al. describes the use of stencils that may be secured to the skin to allow an inexperienced person to apply dye such as henna to the skin. The stencil involves a non-absorbent layer that is secured to the skin with an adhesive layer, and has an opening that forms a pattern in which the henna-based dye may be applied. U.S. Pat. No. 5,470,351 to Ross et al. describes a stencil that may be applied to the skin for forming a suntan tattoo.

U.S. Pat. No. 6,857,935 to Dohan relates to reusable adhesive body apparel, and describes stickers for placement on the body. The stickers may have a large number of designs, images, colors and effects, and may in some embodiments have a metallic color.

It is desirable to provide alternative forms of temporary tattoos, such as temporary tattoos having an interesting decorative effect, particularly a metallic effect tattoo.

### SUMMARY OF THE INVENTION

In a first aspect, the present invention concerns a process for creating a temporary metallic leaf tattoo. The method involves creating a template for the temporary tattoo, applying the template to the skin of a person, applying a skin safe adhesive to the skin within the template, applying metal leaf to the adhesive layer on the skin within the template, and removing the template to reveal a metal leaf tattoo on the skin.

In another aspect, the present invention concerns a process for creating a temporary metallic leaf tattoo. The method involves applying a skin safe adhesive to the skin of a person, and applying metal leaf to the adhesive layer on the skin.

In a preferred arrangement, the template is backed with an adhesive, and is temporarily adhered to the skin. The template may be formed of vinyl.

In another arrangement, a layer of adhesive or coating is applied over the metal leaf tattoo.

The metal leaf may be selected from the group consisting of precious metals, platinum, gold, silver, copper, aluminum and colored metal leaf.

The metal leaf may be applied to the adhesive layer on the skin by using a brush to place the leaf onto the adhesive or by directly placing the leaf on the adhesive.

In some arrangements, multiple applications of metal leaf may be applied to create a multiple toned effect, the method comprising creating a template, applying the metal leaf and removing the template for each different metal leaf.

These and other aspects and features of the invention will become apparent upon review of the enclosed drawings and detailed description below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a back view of a vinyl template for use in the invention.

FIG. 2 is a view showing the vinyl template of FIG. 1 being applied to an application tape layer.

FIG. 3 is a front view showing the vinyl template of FIG. 1 placed on skin, and with its application tape layer being peeled away.

FIG. 4 is a view showing adhesive being applied to the skin within the vinyl template of FIG. 1.

FIG. 5 is a view showing the application of metal leaf to the glued skin within the vinyl template of FIG. 1.

FIG. 6 is a view of a temporary tattoo according to the invention applied to the skin.

### DETAILED DESCRIPTION OF CERTAIN EMBODIMENTS OF THE INVENTION

This invention relates to temporary metallic leaf tattoos that are created using metal leaf. By temporary tattoo is meant any design that is applied to the skin for decorative purposes, and which remains on the skin for a period of time from several hours up to several weeks. A tattoo is different from makeup in that makeup is painted or applied to the skin and may be easily brushed or washed off, whereas a temporary tattoo is either adhered to the skin, or is formed from a dye which penetrates the skin. Metal leaf is a very thin layer of a metal that can be applied to a surface. Metal leafs that may be used include, but are not limited to precious metals such as platinum, gold (including 24 karat surface gold, and 23 karat surface gold leaf standard, 23 karat glass gold leaf standard, 23 karat patent gold leaf standard—the most commonly used gold leaf, 21¾ karat surface gold leaf, 18½ karat surface lemon gold leaf, 18½ karat glass lemon gold leaf, 18½ karat patent lemon gold leaf, 16 karat surface pale gold leaf, 16 karat glass pale gold leaf, 14 karat surface gold leaf and 10 karat gold leaf), variegated leaf green, variegated leaf blue, other colors of variegated and non-variegated leaf, composition leaf #2½, silver leaf domestic, aluminum leaf, and copper leaf.

In one aspect of the invention, a design for a tattoo is first created in order to form a template. The design may be created using a computer-based design program, freehand, or using standard graphics images and/or pre-prepared and commercially available designs such as ClipArt. Any suitable design may be used, but the design is preferably one in which an outline of a shape or the body of a shape may be easily seen. If the design is created freehand, it is preferably converted into a digital art work file for use with a cutting machine for cutting the template. The art work file is fed into a vinyl or other cutting machine, and the cutting machine is operated to create a template for the design. The cutting machine cuts the design into vinyl that is adhered to a paper backing layer, but does not cut the backing layer. Alternatively, the template may be cut freehand, but this is not as desirable as using an automated cutting machine because it is preferable that the edges of the template are as smoothly cut as possible. The types of vinyl that may be used for the templates include, but are not limited to 2 mm (extra strength), 2.5 mm 3 mm, 3.5 mm, premask vinyl, stencil mask and spray mask vinyl. It will be appreciated that vinyl does not need to be used for the template, as any material which can be adhered to the skin and removed may be used. Preferably, the material should be non-porous and flexible. As an alternative to using a vinyl or other material template, food coloring may be added to adhesive and the design may be painted on to the skin freehand where the metallic leaf is to be applied.

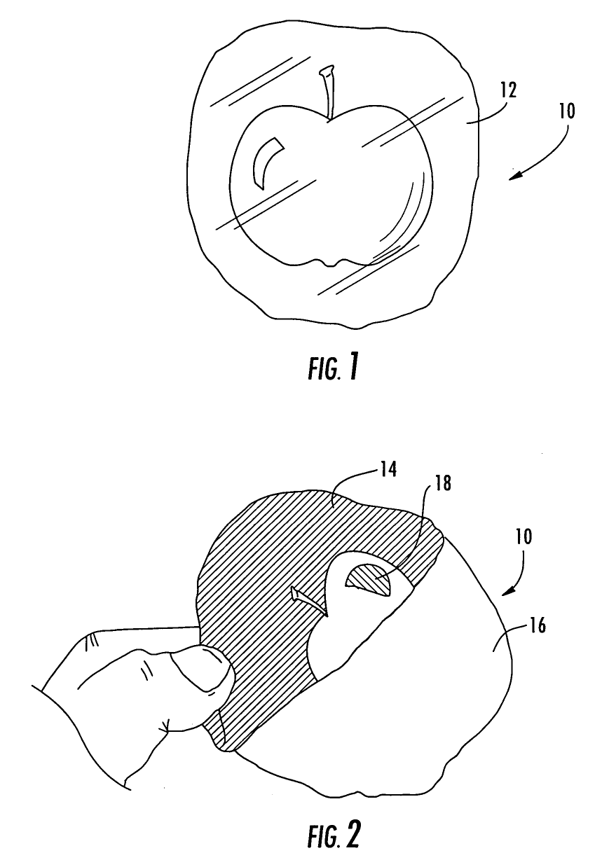

A created vinyl template 10 is shown in FIG. 1. The template 10 includes a paper backing layer 12, and a vinyl layer 14. Once the vinyl template design is cut using a cutting machine, or hand cut with an Exacto type blade or knife, the vinyl is peeled out of the template where the metallic effect is desired, leaving a template outline for the creation of an image. The illustrated template is for an apple, but of course any desired image or design may be used. Small areas 18 of vinyl may be left in the design to create highlights and other shapes. Application tape 16 is applied over the entire vinyl area 14 while it is still applied to its backing layer 12, as shown in FIG. 2. After application, the application tape 16 is squeegeed to remove all air, wrinkles and bubbles.

The area of skin 20 to be tattooed is cleaned with alcohol. The backing paper 12 is removed from the template 10 to reveal adhesive on the back of the vinyl 14 (not shown), and the template 10 is applied firmly to the cleaned area of skin 20. The person applying the template does not need to touch the surface of the vinyl because they can manipulate the template using the application tape layer 16. This keeps the surface of the vinyl clean during application of the template. The application tape 16 is then peeled away to reveal the vinyl template 10, as shown in FIG. 3.

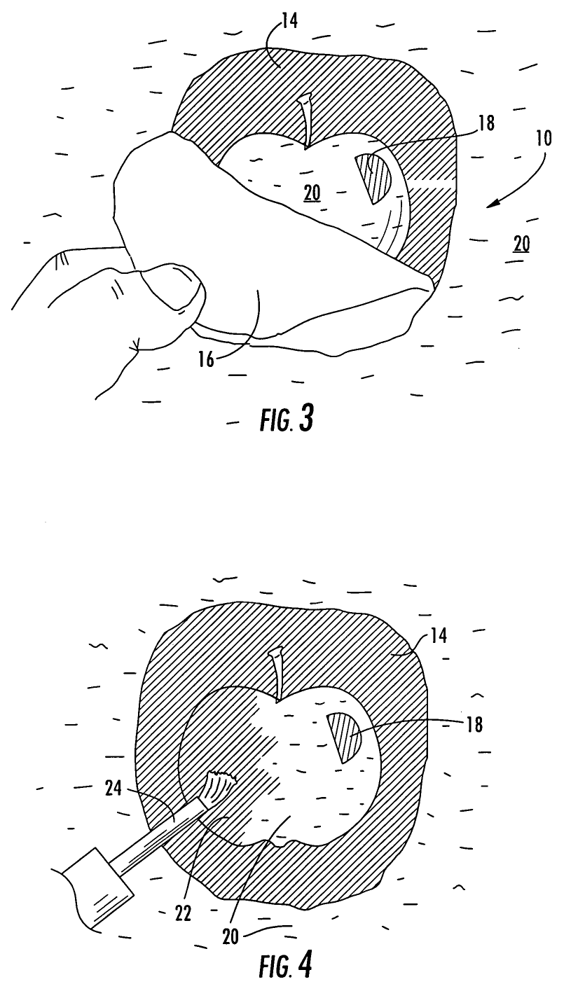

Skin safe adhesive 22 is applied within the template where the metallic leaf is to be adhered to the skin. Any suitable adhesive may be used, such as prosthetic adhesive, spirit gum or medical adhesive, depending on how long the temporary tattoo is intended to last. Different layers of adhesive may also be used, with a layer of talcum powder between the layers in order to provide additional tack. The talcum powder absorbs oil from the skin and helps the temporary tattoo to last longer. The adhesive 22 may be applied to the skin using any suitable method, such as by using an application brush 24, as shown in FIG. 4.

Metal leaf 26 is applied into the template 10 wherever a metallic effect is desired. Metal leaf is readily available for several different metals, such as gold, silver and copper, and is provided in the form of a very thin layer of metal on a backing paper. The leaf can be applied using either of two methods. One method is to apply the leaf directly by pressing it face down onto the adhesive by holding it by the paper backing. With this method, the person applying the leaf must be careful not to touch the metallic side, otherwise the metal leaf will adhere to whatever is touched due to static electricity. Alternatively, an animal hair brush 28 may be used, as shown in FIG. 5. The brush is brushed on a surface or material that will create a static electric charge, such as on the hair of the person applying the metal leaf. The charged brush is then placed just above the metal leaf so that the metal will be lifted from the backing paper due to the static charge. The metal leaf 26 is then transferred to the glued skin by carefully lowering the brush to the glued skin until static electricity from the skin pulls the metallic leaf gently onto the glued area of skin. The metallic leaf can then be manipulated to lay flat. At this time, the surface of the leaf can be manipulated to form a desired surface texture or design. For example, felt wrapped around the thumb of the person applying the tattoo may be used to create a swirl effect on the surface. Each swirl circle can be overlapped until the surface has a machined or brushed metal look, as in burnishing. Any excess metal may be brushed away. The metal leaf is applied until the area within the template is completely covered. Any areas that were missed on the first application may be filled in using a subsequent application so that the metal leaf forms a coating over the glued skin.

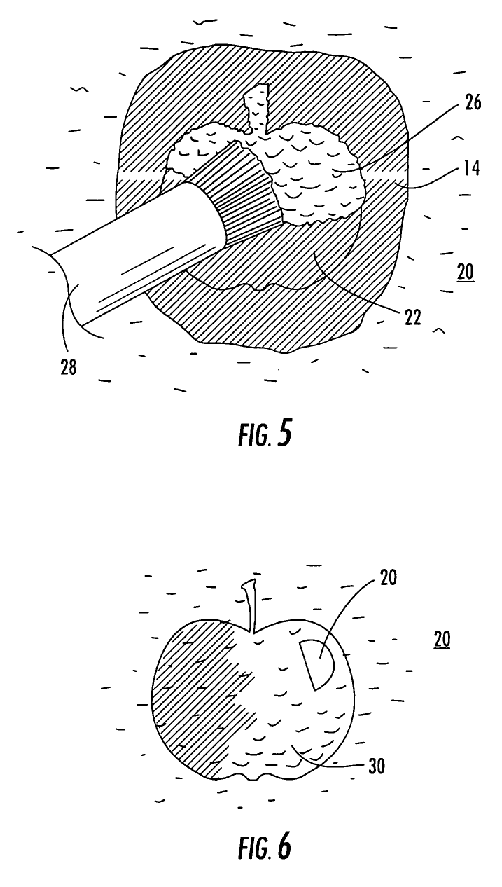

Excess metal leaf 26 is brushed away from the template, and the template is removed slowly from the skin to leave a metal leaf tattoo 30 adhered to the skin 20, as shown in FIG. 6. Preferably, the template is removed smoothly from one comer to the opposite comer. The template must be removed slowly and carefully so that small metal leaf covered areas are not disturbed.

The finished design may be coated with a layer of skin safe adhesive or other coating to create a longer lasting and shiny finish. A suitable adhesive that may be used is Endurabond epoxy adhesive, although it will be appreciated that any suitable adhesive or coating may be used. The temporary tattoo 30 will stay on the skin for at least a week.

Multiple metal leafs may be used to create a two-tone effect. For example, a gold outline to a silver image can be created. The process for creating a two-tone effect is the same as for creating a single color metallic leaf tattoo, but is carried out multiple times. The central part of the design is created first, for example, a template may be created for a silver body of the tattoo, which is applied to the skin before gluing, and the silver leaf applied to the adhesive. A gold outline may be applied either free hand or by using a template larger than the silver body, leaving a space between the already applied silver and the template for the gold outline so that the vinyl template for the gold area is not placed over any silver leaf. The silver body can be used as a guide for putting on the adhesive. The gold is applied, and the template is removed after any excess gold has been brushed away. It will be appreciated that a multiple tone effect can be created with any combination of metals or any order of metal leaf application. Leaf inlays may also be used, either in place of the central template design, or to add an additional tone effect. A leaf inlay would be added freehand without using a template.

The foregoing is provided for purposes of illustrating, explaining, and describing embodiments of this invention. Modifications and adaptations to these embodiments will be apparent to those skilled in the art and may be made without departing from the scope or spirit of this invention. All publications and patents mentioned herein are incorporated by reference in their entirety, including all figures, graphs, equations, illustrations, and drawings to describe and disclose specific information for which the reference was cited.

1. A method of creating a temporary tattoo, the method comprising the steps of:
creating a template for the temporary tattoo;
applying the template to the skin of a person;
applying a skin safe adhesive to the skin within the template;
applying metal leaf to the adhesive layer on the skin within the template; and
removing the template to reveal a metal leaf tattoo on the skin.

2. The method according to claim 1, wherein the template is backed with an adhesive, and is temporarily adhered to the skin.

3. The method according to claim 1, wherein the template is formed of vinyl.

4. The method according to claim 1, wherein a layer of adhesive or coating is applied over the metal leaf tattoo.

5. The method according to claim 1, wherein the metal leaf is selected from the group consisting of precious metals, platinum, gold, silver, copper, aluminum and colored metal leaf.

6. The method according to claim 1, wherein the metal leaf is applied to the adhesive layer on the skin using a brush to apply the leaf onto the adhesive or by directly placing the leaf on the adhesive.

7. The method according to claim 1, wherein multiple applications of metal leaf are applied to create a multiple toned effect, the method comprising creating a template, applying the metal leaf and removing the template for each application of metal leaf.

8. A process for creating a temporary metallic leaf tattoo comprising the steps of:
applying a skin safe adhesive to the skin of a person; and
applying metal leaf to the adhesive layer on the skin.
